(19) United States
(12) Patent Application Publication
(10) Pub. No.: US 2009/0005880 A1
(43) Pub. Date: Jan. 1, 2009

(54) Method for Preparing Endosseous Implants Anatase Titanium Dioxide Coating

(75) Inventors: Francesco Carinci, Bologna (IT); Carlo Alberto Bignozzi, Ferrara (IT); Claudio Minero, Racconigi (IT); Valter Maurino, Pinerolo (IT)

Correspondence Address: COLEMAN SUDOL SAPONE, P.C., 714 COLORADO AVENUE, BRIDGE PORT, CT, 06605-1601, US

(73) Assignee: GUYA BIOSCIENCE S.r.l., Ferrara (IT)

(21) Appl. No.: 11/577,514

(22) PCT Filed: Oct. 13, 2005
(86) PCT No.: PCT/IB2005/003155

§ 371 Date: Aug. 22, 2008

(30) Foreign Application Priority Data
Oct. 22, 2004 (IT) BO2004A 000653

(51) Int. Cl. A61F 2/28; A61F 2/02; A61B 19/00

(52) U.S. Cl. 623/23.6; 623/16.11; 623/11.11; 128/898

(57) ABSTRACT

The method includes the following steps: formulation of liquid, non-gelled and stable precursor by solvolysis of Ti(IV) compounds; precursor deposition on endosseous implant surface; thermal treatment to achieve film densification, in the presence of oxygen, of a complex formed by the above mentioned endosseous implant and precursor, to obtain on the implant surface a thin film of nanocrystalline titanium dioxide with good mechanical and chemical stability. The complex above, under a persistent W irradiation modify its surface status conferring a sensible increasing of wettability chemical and biological decontamination.

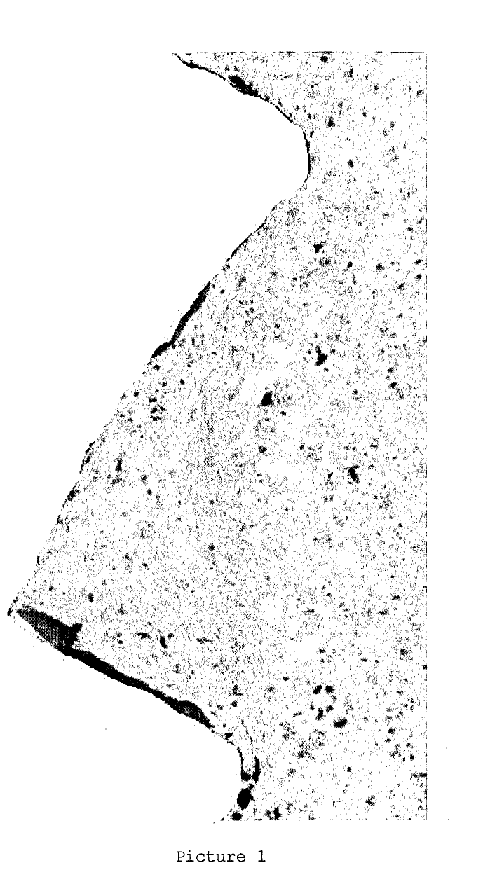

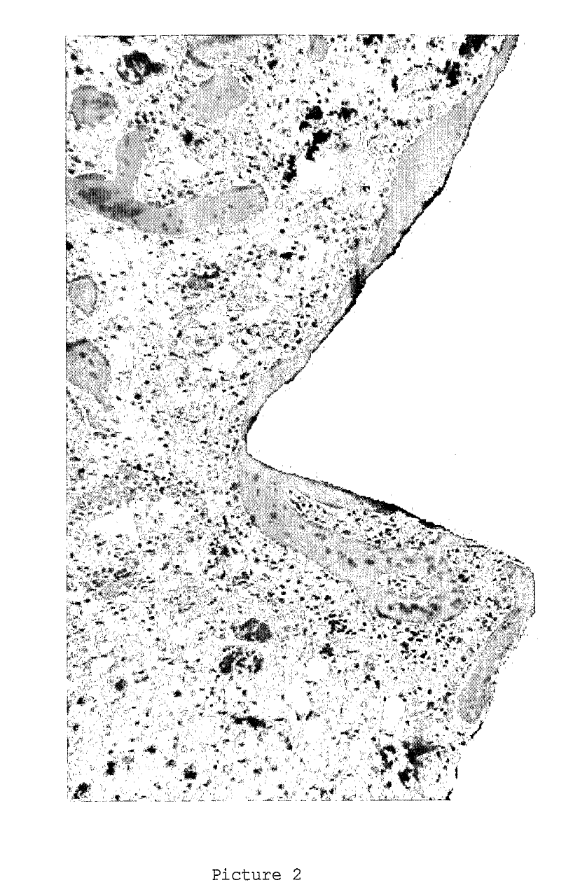

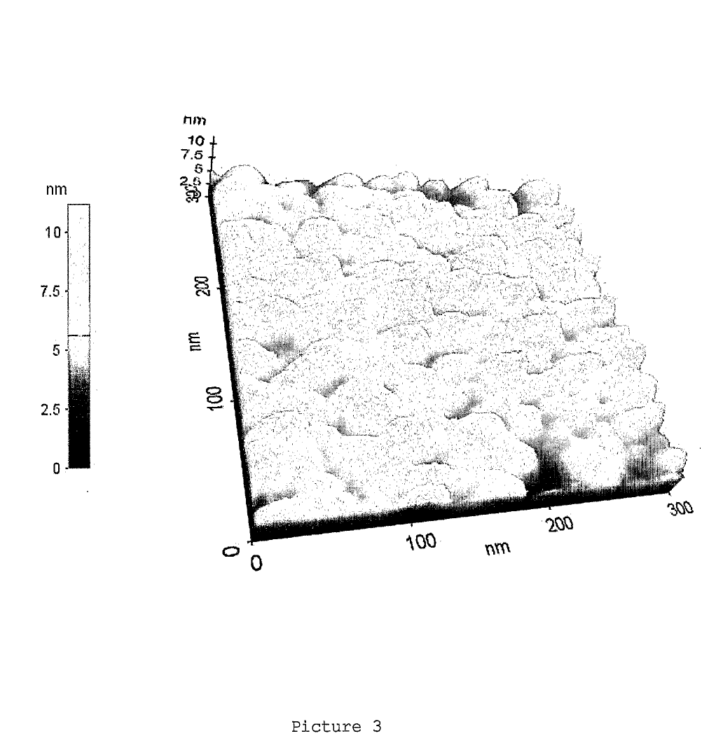

### TECHNICAL FIELD OF THE INVENTION

The invention relates to the technical field concerning the preparation of endosseous implants with high osseointegration degree, and in particular the invention concerns a method for preparing endosseous implants with high osseointegration degree by means of titanium dioxide coating thin film in the anatase crystalline form.

### PRIOR ART

It is well known that titanium is widely used for medical purposes for its mechanical properties and its biocompatibility. Biological compatibility can be detected not only in the absence of inflammatory rejection crisis, but also in the increase of biological process of the receiving tissue, in the case of endosseous implants is expressed in an increased osseointegration.

There are two crystalline forms of titanium dioxide: rutile and anatase, the former is the most thermodynamic stable.

Since sixties oral, maxillofacial and orthopedics surgeon have been using oral implants, screws, patches and artificial prosthesis, in order to replace lost teeth, fix fracture and for pathological articulations replacement. Moreover titanium is used in manufacture of several types of surgical instruments such as trepanning saws and spatulas.

In the last thirty years titanium biocompatibility has been demonstrated, in several scientific report concerning in vitro studies, in vivo models and clinical trials.

Concerning dental implants used as root of lost teeth substitutes and in the orthopedics reconstructive surgeon, titanium biocompatibility is show in its capability to determine osseointegration. Osseointegration of a fixture in bone is defined as the close apposition of new and reformed bone in congruence with the fixture. When process is completed a direct, structural and functional, connection is established, capable of carrying normal physiological loads without excessive deformation and without initiating rejecting mechanisms. The process intervenes in a lapse of time of sixty days, comparable to the fracture fixing process. This period can differ occurring in other variables such as: microgap dimension among implant and bone, primary implant stability, type of implant surface, etc.

Osseointegration may depend on some specific implant features: a) type of material, b) macroscopic surface design (i.e. screw design in root-form dental implants), c) type of surface. Factor c) is determined according to the manufacturing technique adopted, for example sand blasted acid atched, anodizated, grit-basted and passivated ecc. The surface is important for creation of implant surface microroughness is needed for filopodi osteoblast anchorage. However factor a) is the most important to determine osseointegration. In fact during sixties iron made implants were used. Branemark together with other scientists (Branemark P I, Adell R, Breine U, Hansson B O, Lindstrom J, Ohlsson A. “Intra-osseous anchorage of dental prostheses. I. Experimental studies.” Scand. J. Plast. Reconstr. Surg. 1969; 3(2):81-100. Adell R, Hansson B O, Branemark P I, Breine U. “Intra-osseous anchorage of dental prostheses. II. Review of clinical approaches.” Scand. J. Plast. Reconstr. Surg. 1970; 4(1):19-34) demonstrated with their studies that, differently from iron, titanium is capable of stimulate osteogenesis. Since then all endosseous implants are made of titanium.

Metallic titanium oxidize on the surface. Surface oxidation process makes titanium biocompatible. Transitional metals belonging to group IIIA and IVA, and their leagues, preferentially containing titanium, whose surface is at least partially converted in oxide, and whose surface is composite and preferentially include calcium phosphate, preferentially formed through Plasma Electrolytic Oxidation, show a good biocompatibility (WO03094774).

Titanium surface modification by means of grit-blasting with titanium dioxide particles, preferentially with 1-50 micron diameter, do not introduce polluting material and create the required corrugated surface (U.S. Pat. No. 5,667,385) necessary to improve the linkage implant-bone tissue.

Recent studies related to the absorption of human plasma fibronectin, has demonstrated that wettability and titanium oxide surface charge have an important orle in the protein absorption (D. E. MacDonald, N. Deo, B. Markovic, M. Stranick, P. Somasundaran, “Absorption and dissolution behavior of human plasma fibronectin on thermally and chemically modified titanium dioxide particles”, Biomaterials 23 (2002)1269-1279).

Surfaces coated with titanium dioxide show anti-bacterical and self-cleaning characteristics, related to photocatalytic properties of titanium dioxide, in the anatase form, widely discussed and documented in scientific literature (E. Pelizzetti, N. Serpone, E. Pramauro, M. Barbeni, E. Borgarello, M. Graetzel, Nouv. J. Chim. 1984, 8, 547-550; E. Pelizzetti, N. Serpone, “Heterogeneous Photocatalysis”, J. Wiley and Sons 1989; E. Pelizzetti, C. Minero, V. Maurino, “Adv. Colloid and Interf Sci.” 1990, 32, 271-316; D. F. 011 is, H. Al-Ekabi, “Photocatalytic Purification and Treatment of Water and Air”, Elsevier, Amsterdam, 1993; E. Pelizzetti, C. Minero, “Mechanism of the photooxidative degradation of organic pollutants over titanium dioxide particles”, Electrochim. Acta, 38, 47-55, 1993).

It is also well known that materials with photocatalytical properties are also characterized by capability of mineralization organic compounds under light irradiation of present in contact solution or absorbed as pollutant on their surface, and to denature bacteria as show in the Escherichia Coli case study by K. Sunada, Y. Kikuchi, K. Hashimoto, A S. Fujishima, “Bactericidal and Detoxification Effects of TiO2 Film Photocatalysts” Environ. Sci. Technol., 1998, 32, 726.

This latter property is important for the purpose of sterilization of dental implants, or endosseous implants in general, before their surgical utilization. This could be done by immersion of implants in water, preferentially de-ionized, followed by ultraviolet light irradiation (230-380 nm, preferentially 250-320 nm) before use.

On a surface fully anatase coated, which shows an elevate photocatalytical activity, under a persistent UV irradiation modify its surface status conferring a super-hydrophilic property, consisting in a sensible increasing of wettability from the surface water itself (the contact angle with the film gradually decreases to 0 degree) and can foster biocompatibility. Both positive properties can be obtained under ultraviolet light irradiation before use.

Preparations of sols precursors that can be imagined from the technical scientific and patented literature are not usually stable in time, may gel and settling down solid particles becoming useless for industrial preparations, or producing coating films of insufficient optical and aesthetic quality either for transparency or homogeneity. The resistance of these films to abrasion and chemical agents is often unknown.

The deposition on the materials concerned in the present invention previously prepared powders of titanium dioxide (see for example the EP patent publications Nos. 792687A1, 684075A1, 866101A1) usually leads to coated materials that are not resistant to abrasion, either inorganic melting agents or polymers, often having adverse effects on the catalyst activity, its performance in the photocatalytic process, and limiting its efficacy toward its final scope. Film mechanical resistance is an essential requirement for the placement of dental implants, or endosseous implants in general, basing biocompatibility on surface treatment. Deposition by means of Metal Organic Chemical Vapor Deposition forms a dioxide titanium layer in the anatase form crystallographic oriented, improving implant hisomorphic parameter and microhardness of the new formed bone. (Giavaresi G., Ambrosio L., Battiston G. A, Casellato U., Gerbasi R., Finia M., Aldini N, N., Martini L., Rimondini L., Gerbino R., “Histomorphometric, ultra structural and microhardness evaluation of the osseointegration of nanostructured titanium oxide coating by metal-organic chemical vapour deposition: an in vivo study”, Biomaterials, 2004, 25, 55-83, and patent ITVE 2001A000051).

The procedure MO-CVD requires a dedicate build equipment, and has some inherent applicability limits.

### DESCRIPTION OF THE INVENTION

The object of the present invention is to propose a method for preparing endosseous implants with high osseointegration degree.

Another object of the present invention is to propose a coating method on metallic supports with thin titanium dioxide photocatalytic film, in the anatase nanocrystalline form, fostering osseointegration.

A further object of the present invention is to propose a coating method using a film stable on the surface of the treated implant, with a film having very good mechanical properties.

A still further object of the present invention is to propose a coating method on endosseous implants of several shapes, even irregular and with internal gaps.

Moreover it must be added the intention to propose a method characterized by simple and quick phases.

The above mentioned object are obtained in accordance with the contents of claims, by a method for preparing endosseous implants with high osseointegration degree by means of titanium dioxide coating thin film in the anatase crystalline form, including the following steps:

formulation of liquid, non-gelled and stable precursor by solvolysis of Ti(IV) compounds;

precursor deposition on endosseous implant surface;

thermal treatment to achieve film densification, in the presence of oxygen, of a complex formed by the above mentioned endosseous implant and precursor, to obtain on the implant surface a thin film of nanocrystalline titanium dioxide with good mechanical and chemical stability;

the complex above, under a persistent UV irradiation modify its surface status conferring a sensible increasing of wettability chemical and biological decontamination.

### BRIEF DESCRIPTION OF THE PICTURES

The characteristics features of the invention are pointed out in the following with particular reference the enclosed picture, where are described some results obtained through adoption of the proposed method:

picture 1 shows an example of osteogenesis stimulation on uncoated surface;

picture 2 shows an example of osteogenesis stimulation on titanium dioxide fully coated surface, in the anatase nanocrystalline form, produced according to the method describe in this invention;

picture 3 shows an anatase surface nanoscopic topography (300×300 nm), produced according to the method describe in this invention. This measure is obtained through a no-contact atomic force microscopy. Vertical scale is not isotropic if referred to plane scales.

### PREFERRED EMBODIMENTS OF THE INVENTION

The process of the disclosed invention is in several phases, and concerns the formulation of liquid, non-gelled and stable precursor made of inorganic or metal-organic compounds of Ti(IV). After deposition on the support, using a simple technique (such as immersion and extraction at a controlled speed—dip-coating process) followed by a thermal treatment to achieve densification, is formed a thin coating films in anatase crystalline form, firmly anchored on dental implants surface.

The method set in this invention consist of the deposition on the metallic dental implant support or endosseous implants in general, of a stable liquid precursor made of inorganic or metal-organized, compounds of Ti(IV) partially or totally hydrolyzed, and surfactants, and/or acids, and a suitable organic doping, in particular s-triazine derivates, included to improve the biocompatibility, the photocatalytic activity and mechanical resistance. Then the dental endosseous implant is undergone to a thermal treatment to achieve film densification, then activation and sterilization is performed, by means of ultraviolet light irradiation of the dental implant surface, or in general endosseous implant, (230-380 nm, preferentially 250-320 nm) for at least 24 hours before the surgical use.

Below are described formulations and procedures related to the method set in the disclosed invention.

The first phase consists in the formulation of the non-gelled liquid precursor comprising titanium (IV) alkoxides at concentrations in the range 0.1% to 35% by weight. The liquid precursor is stable in air and can be stored for several months without being subject to alteration. The non-gelled liquid precursor contain Ti (IV) compounds containing in their formulation alkoxides, and in particular tetrabutoxy-ortho-titanate, tetrapropoxy-ortho-titanate, tetraisopropoxy-ortho-titanate, or halides, in particular the tetrachloride, or other kind of complexes like bis(ammonium lactate) dihydroxide titanium (IV). Solvolysis of Ti(IV) compounds needs from 1 minute to 36 hours, at temperatures ranging from 5° C. to the solvent boiling point, eventually under pressure (1-20 atm) at temperatures ranging from 0° C. to 120° C., eventually doped with a selected s-triazine derivative, and/or urea, and/or dicyandiamide. The solvolysis is necessary to form compounds of Ti(IV) that are less volatile than the original compounds, unable to vaporize during the subsequent thermal treatment, and showing good film sticking properties, and sufficient thickness to the support. Otherwise, the precursor could be partially or completely vaporized and lost during the thermal treatment, with formation of irregular and/or discountinous or no coatings. The water concentration needed by the hydrolysis ranges from 0.1 to 30% by weight, in presence of organic solvents. Addition of organic solvents can be avoided if the coating procedures (dip, spray or roll coating) allow it. In this case water concentration in the formulations can reach up to 96% by weight. Organic solvents, which are alcohols, also polyfunctional and containing oxygen in ether bonds, carry 1-10 carbon atoms and 1-6 oxygen atoms, or lactones containing 4-6 carbon atoms, or mixtures thereof in all proportions.

The solvent choice is made according to procedures used for deposition (dip-coating, spray or roll-coating) and the titanium dioxide film layer thickness requested. Sol precursors obtained without addition of organic solvent, can be subjected to dialysis to reduce concentration of electrolytes and substances flocculants molecular weight less than 1000 uma. The gelation of the liquid precursor, either contemporary to the preparation step or when the precursor is stored before deposition renders it incompatible with the deposition with dip-coating, spray or roll-coating, especially if thin films below 10 μm are desired. To avoid gelation an inorganic or organic acid is added at concentrations ranging from 0.10 to 20% by weight and/or a surfactant of type nonionic, or cationic, or anionic, or zwitterionic and their mixtures in all proportions, at concentrations ranging from 0.10 to 10% by weight. Non-ionic surfactants alkyl- or alkylarylethoxilate and their mixtures in all proportions (for example the commercial products Brij 30, Brij 35, Triton X100), and/or alkyl or alkylethoxysulphate anionic surfactants (for example sodium dodecyl sulphate), and/or alkylbenzene sulphonate, and/or cationic surfactants, e.g. cetyltrimethylammonium bromide, and/or zwitterionic surfactants, like betaine derivates, are among the surfactants useful to block the gelation and TiO₂ particle growth. Among inorganic acids the following are suitable: nitric acid, sulfuric acid, phosphoric acid, hydrofluoric acid, hydrochloric acid, perchloric acid and their mixtures in all proportions. Among organic acids are adequate those with linear or branched chains, also with 2 or 3 carboxylic groups and/or containing hydroxyl-, and/or chloro-, and/or fluoro-, and/or bromo-groups, or benzoic acid and its derivates, and/or other carboxylic acid with aromatic structure. The presence of surfactant and/or the acid as the additional effect of inhibiting the formation in the liquid precursor of titanium dioxide particles exceeding the critical diameter threshold estimated in 150-200 nm: beyond this dimension particles will form coating films less resistant to the abrasion and less uniform. The gelation processes and formation of particulate titanium dioxide are inhibited by the presence of the acid and/or the surfactant at temperatures ranging from −10° C. to 100° C. At ambient temperature (not higher than 30° C.) the disclosed formulation renders the precursor stable against gelation and particle formation and settling for 6 to 12 months, depending on the composition. The coating with the precursor made by the above cited procedures, is followed by a thermal treatment lasting 10-200 min at temperatures ranging from 300° C. to 800° C., in the presence of a gas phase containing oxygen in the range 1% to 50% by volume, in order to fully convert the precursor in microcrystalline anatase TiO₂, and obtain a coating with good mechanical and chemical stability.

Table 1 shows the best mode of carrying out the invention, by one example of the precursor used for the coating, according to the disclosed formulation. Sometimes the preparation has to be performed under nitrogen atmosphere, depending on the organic solvent.

A biological test performed on animal model consist in bone grafts two implant series (coated and uncoated) in rabbit tibia. Animals are sacrificed after 30 days and the block section, containing the implant, is retrieved for histomorfometric analysis evaluation.

The golden standard for the biological test consisting in bone grafts alloplastic material in rabbit femur/tibia, is the system internationally recognized for biocompatibility trials (Scarano A, Di Carlo F, Quaranta M, Piattelli A. “Bone response to zirconia ceramic implants: an experimental study in rabbits.” J. Oral Implantol. 2003; 29(1):8-12. Piattelli M, Scarano A, Paolantonio M, Iezzi G, Petrone G, Piattelli A. “Bone response to machined and resorbable blast material titanium implants: an experimental study in rabbits.” J. Oral Implantol. 2002; 28(1):2-8. Cordioli G, Majzoub Z, Piattelli A, Scarano A. “Removal torque and histomorphometric investigation of 4 different titanium surfaces: an experimental study in the rabbit tibia.” Int. J. Oral Maxillofac. Implants. 2000 September-October; 15(5):668-74. Piattelli A, Scarano A, Di Alberti L, Piattelli M. “Histological and histochemical analyses of acid and alkaline phosphatases around hydroxyapatite-coated implants: a time course study in rabbit.” Biomaterials. 1997 September; 18(17):1191-4).

TABLE 1
Example of anatase film coating according
to the herein disclosed procedure.

| | | Preferred |
|---|---|---|
| Component | Weight % | Weight % |
| Step 1: Liquid precursor formulation | | |
| Isopropyl alcohol | 46 to 90 | 81 |
| Phosphoric acid 85% | 0.1 to 10 | 0.8 |
| Water | 0.2 to 5 | 2.8 |
| Brij 56 | 1 to 20 | 3.4 |
| Tetraproxy-ortho-titanate | 10 to 20 | 12 |

| Step 2: Solvolysis: closed container from 24 h at 60° C. | |
|---|---|
| Step 3: Coating and film densification | |
| Deposition procedure | Dipcoating, rate 12 cm min⁻¹ |
| Support | Titanium or other metallic material |
| Thermal treatment | 550° C. for 30 min under forced air flow |

From experimental data, as shown in histological pictures 1 and 2 at 30 days, coated surface demonstrated a clear neosteogenesis stimulation. Picture 2 shows a 75% increasing of bone tissue (red colored area) if compared to uncoated specimen in picture 1, improve osteogenesis of the anatase entirely coated surface. Coatings made with the disclosed method form thin anatase film of 0.02-10 μm thickness, according to conditions of the sol procedure deposition. The film show resistance to abrasion and chemical agents, it is homogeneous and covered at a microscopical level. In picture 3 is shown a film topography obtained adopting the disclosed method. It can be marked anatase particles with 30-50 nm diameters, collapsed to create a compact film with a superficial local roughness within few nm (see the picture vertical scale).

The described method allow to obtain titanium dioxide coating thin films in the anatase crystalline form.

This coating film shows an elevate photocatalytical activity improving osseointegration. The disclosed invention concerns the formulation of liquid, non-gelled and stable precursors for a low cost manufacturing coating film process (dip-coating, spray or roll-coating). The claimed method allows avoiding the gelification of the precursor, running away the need of further re-peptization of the gel as usually required in common sol-gel methods. The liquid precursor is stable in air, and storable for some months without alteration. The film obtained according to the procedure herein shows very good mechanical properties, adhesion to the metallic support and abrasion resistance without the intervention of an in-between layer. The claimed method, adopting a thermal treatment to achieve the film densification, lead to the formation of an anatase film layer, improving mechanical and chemical stability. Moreover the deposition process can be applied to a large number of dental implants, or endosseous implants in general, applied on a proper material support allowing film deposition, for instance by means of immersion and extraction of the support at a controlled speed.

The disclosed method allow to manufacture thin coating films in anatase crystalline form, firmly-anchored onimplants surface, showing the following strengths: improves osseointegration; confers super-hydrophilic and anti-bacteria properties to the surface irradiated with ultraviolet light in a wavelength at 230-380 nm, preferentially 250-320 nm; allows to manufacture endosseous implants with nanocrystalline anatase entirely coated supports of materials different from titanium (i.e. iron).

The dental endosseous implant, or in general endosseous implant, coated using a solution of Ti(IV) followed by a thermal treatment as indicated in the disclosed method, improve osteogenesis of the anatase entirely coated surface, it shows anti-bacteria and self-cleaning properties to the surface irradiated with ultraviolet light. The disclosed method shows a further advantage consisting in a thin coating films, in anatase nanocrystalline form, showing outstanding mechanical, super-hydrophilic and photocatalytic activity properties, at a low cost procedure.

Surface coated following the disclosed method is capable of improving new bone apposition, which represents a key factor in the definition of prosthesis biocompatibility. A further strength of the disclosed invention, differently from precursors prepared according to other common sol-gel methods, is to set a coating method on endosseous implants of several shapes, even irregular and with internal gaps. It does not need expensive deposition equipment and produce a greater osseointegration in endosseous implants. The disclosed method allows avoiding the gelification of the precursor, running away the need of further re-peptization of the gel as usually required in the common sol-gel methods.

It is understood that what above, has been described as a pure, not limiting example, therefore, possible practical applications variants of the proposed steps remain within the protective scope of the invention, as described above and claimed hereinafter.

1. Method for preparing endosseous implants with high osseointegration degree by means of titanium dioxide coating thin film in the anatase crystalline form, characterized in that it includes the following steps:
formulation of liquid, non-gelled and stable precursor by solvolysis of Ti(IV) compounds;
precursor deposition on endosseous implant surface;
thermal treatment to achieve film densification, in the presence of oxygen, of a complex formed by the above mentioned endosseous implant and precursor, to obtain on the implant surface a thin film of nanocrystalline titanium dioxide with good mechanical and chemical stability;
the complex above, under a persistent UV irradiation modify its surface status conferring a sensible increasing of wettability chemical and biological decontamination.

2. Method, according to claim 1, characterized in that said titanium dioxide is in the anatase nanocrystalline form.

3. Method, according to claim 1, characterized in that said liquid non-gelled precursor includes:
a titanium (IV) compound at concentrations expressed as titanium dioxide equivalent, in the range 0.1% to 35% by weight of the liquid precursor;
water at concentrations in the range 0.1% to 30% by weight;
an organic solvent;
an organic or mineral acid and their mixtures, at concentrations in the range 0.1% to 20%, avoiding the gelification of the precursor;
a surfactant of type nonionic, or cationic, or anionic, or zwitterionic and their mixtures in all proportions, at concentrations ranging from 0.1% to 10% by weight.

4. Method, according to claim 1, characterized in that said liquid non-gelled precursor includes:
a titanium (IV) compound at concentrations expressed as titanium dioxide equivalent, in the range 0.1% to 30% by weight of the liquid precursor;
water at concentrations up to 96% by weight;
an organic solvent;
an organic or mineral acid and their mixtures, at concentrations in the range 0.1% to 20%, avoiding the gelification of the precursor;
a surfactant of type nonionic, or cationic, or anionic, or zwitterionic and their mixtures in all proportions, at concentrations ranging from 0.1% to 10% by weight.

5. Method, according to claim 1, characterized in that said film entirely coats endosseous implant surface.

6. Method, according to claim 1, characterized in that said solvolysis of Ti(IV) compounds needs from 1 minute to 36 hours.

7. Method, according to claim 3, characterized in that said solvolysis is performed at concentrations ranging from 0° C. and solvent boiling point.

8. Method, according to claim 1, characterized in that said solvolysis of Ti(IV) compounds needs from 1 minute to 36 hours and it is performed at concentrations ranging from 0° C. and solvent boiling point.

9. Method, according to claims 1, characterized in that said solvolysis of Ti(IV) is performed at temperatures ranging from 0° C. to 120° C., under pressure (1-20 atm).

10. Method, according to claim 1, characterized in that said precursor deposition is performed by means of coating procedures such as dip-coating, spray-coating or roll coating.

11. Method, according to claim 1, characterized in that the presence of oxygen during the thermal treatment is in the range 1% to 50% by volume.

12. Method, according to claim 1, characterized in that said thermal treatment is performed at temperatures ranging from 300° C. to 800° C.

13. Method, according to claim 1, characterized in that said thermal treatment is performed at temperatures ranging from 300° C. to 800° C., in the presence of a gas phase containing oxygen in the range 1% to 50% by volume.

14. Method, according to claim 1, characterized in that said ultraviolet light are in a wavelength at 230-380 nm.

15. Method, according to claim 1, characterized in that said ultraviolet light are in a wavelength at 250-320 nm.

16. Method, according to claim 1, characterized in that said ultraviolet light irradiation must stay for at least 30 minutes.

17. Method, according to claim 3, characterized in that said compounds contain in their formulation tetrabutoxy-ortho-titanate.

18. Method, according to claim 3, characterized in that said compounds contain in their formulation tetrapropoxy-ortho-titanate.

19. Method, according to claim 3, characterized in that said compounds contain in their formulation tetraisopropoxy-ortho-titanate.

20. Method, according to claim 3, characterized in that said compounds contain in their formulation titanium tetrachloride.

21. Method, according to claim 3, characterized in that the above mentioned compounds contain in their formulation at least a complex compound.

22. Method, according to claim 21, characterized in that the said compounds contain in their formulation bis(ammonium lactate) dihydroxide titanium (IV).

23. Method, according to claim 3, characterized in that said organic solvent, includes alcohol, polyfunctional and containing oxygen in ether bonds, carrying 1-10 carbon atoms.

24. Method, according to claim 1, characterized in that said phase of liquid precursor deposition followed by a thermal treatment is repeated a predetermined number of times.

25. Method, according to claim 3, characterized in that said precursor includes one transitional element at least.

26. Method, according to claim 3, characterized in that said precursor include one transitional element belonging to group IVA, in an atomic proportion with Ti up to 30%.

27. Method, according to claim 5, characterized in that said solvolysis of Ti(IV) compounds needs from 1 minute to 36 hours and it is performed at concentrations ranging from 0° C. and solvent boiling point.

28. Method, according to claim 6, characterized in that said solvolysis of Ti(IV) compounds needs from 1 minute to 36 hours and it is performed at concentrations ranging from 0° C. and solvent boiling point.

29. Method, according to claim 6, characterized in that said solvolysis of Ti(IV) is performed at temperatures ranging from 0° C. to 120° C., under pressure (1-20 atm).

30. Method, according to claim 7, characterized in that said solvolysis of Ti(IV) is performed at temperatures ranging from 0° C. to 120° C., under pressure (1-20 atm).

31. Method, according to claim 4, characterized in that said compounds contain in their formulation tetrabutoxy-ortho-titanate.

32. Method, according to clam 4, characterized in that said compounds contain in their formulation tetrapropoxy-ortho-titanate.

33. Method, according to claim 4, characterized in that said compounds contain in their formulation tetraisopropoxy-ortho-titanate.

34. Method, according to claim 4, characterized in that said compounds contain in their formulation titanium tetrachloride.

35. Method, according to claim 4, characterized in that the above mentioned compounds contain in their formulation at least a complex compound.

36. Method, according to claim 4, characterized in that said precursor includes one transitional element at least.

37. Method, according to claim 4, characterized in that said precursor include one transitional element belonging to group IVA, in an atomic proportion with Ti up to 30%.
